(19) United States
(12) Patent Application Publication
(10) Pub. No.: US 2010/0145176 A1
(43) Pub. Date: Jun. 10, 2010

(54) Universal Electrode Array for Monitoring Brain Activity

(76) Inventor: David M. Himes, Seattle, WA (US)

Correspondence Address: NEUROVISTA / SHAY GLENN, 2755 CAMPUS DRIVE, SUITE 210, SAN MATEO, CA, 94403, US

(21) Appl. No.: 12/630,300

(22) Filed: Dec. 3, 2009

Related U.S. Application Data
(60) Provisional application No. 61/119,974, filed on Dec. 4, 2008.
(60) Provisional application No. 61/145,098, filed on Jan. 15, 2009.

(51) Int. Cl. A61B 5/0478; A61B 5/0476; A61B 5/0482

(52) U.S. Cl. 600/378; 600/545; 600/544

(57) ABSTRACT

Methods of monitoring brain activity signals in a patient are described, including the steps of identifying a lobe or lobes of the patient's brain in which the patient's seizures originate; selecting an electrode array from a plurality of predetermined dispersed electrode patterns based on the identified lobe or lobes of the brain; implanting the electrode array within the patient's cranium to place the electrodes in contact with the identified lobe or lobes of the brain; and coupling the electrodes to an seizure advisory system. Also described is a seizure advisory system that includes an electrode array having fewer than 32 electrodes in a predetermined dispersed radial pattern, adapted to be implanted through a single opening in the skull, and to be deployed in the predetermined dispersed radial pattern to detect a brain activity signal. The system also includes a communication assembly and an external assembly.

### CROSS-REFERENCE TO RELATED APPLICATIONS

This application claims the benefit of U.S. Provisional Application No. 61/119,974, filed Dec. 4, 2008, and U.S. Provisional Application No. 61/145,098, filed Jan. 15, 2009, which are incorporated herein by reference in their entireties.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

The present invention relates generally to electrode array patterns, placements of electrode arrays and methods of monitoring brain activity. More specifically, the present invention relates to predetermined electrode array patterns for monitoring brain activity signals for monitoring a patient's propensity for seizures.

Electrode arrays are used in a number of different medical procedures for monitoring neurological signals (such as electroencephalographic signals) and/or delivering neuromodulation therapy to the patient's neural tissue (e.g., the brain). Such electrodes may be positioned above the scalp, in contact with a cortical surface of the brain, or deep within the brain's tissue. Electrical signals that are monitored from scalp electrodes are referred to as electroencephalogram signals, while electrical signals monitored using intracranial electrodes are referred to as electrocorticogram. The term “EEG” is used herein to encompass both electroencephalogram and electrocorticogram.

In the hospital setting, for patients who have epilepsy or who are thought to have epilepsy, conventional examination methods typically involve non-invasive monitoring (e.g., scalp electrodes, dense electrode arrays, functional magnetic resonance imaging (fMRI), single photon emission computed tomography (SPECT), computed tomography (CT), MRI, etc.) and/or invasive monitoring (e.g., intracranial electrode arrays) to monitor the patient's brain. Such monitoring may be used to determine (1) whether or not the patient has epilepsy, (2) the type of epilepsy, and/or (3) localization of the seizure focus. The localization of the seizure focus is an important step in determining the portion of the patient's brain that is responsible for the generation of the seizure. This localization procedure is especially important as it relates to epilepsy brain resection surgery. In resection surgery a portion of the brain is resected, or cut away, to remove the seizure focus. Therefore prior to such surgery it is important to localize or pin-point the seizure focus in order to ensure removing the brain tissue that contains the seizure focus without causing damage to areas of the brain responsible for vital functions, such as movement, sensation, language and memory.

Dense electrode arrays typically have 256 (or more) electrodes, usually spaced 10 mm apart (yielding an electrode density of 1 electrode per square centimeter), and are applied to the patient's scalp. Intracranial monitoring typically requires placement via a craniotomy of up to 128 electrodes below the dura and in contact with the cortical surface of the patient's brain.

### SUMMARY OF THE INVENTION

For ambulatory monitoring using implanted electrodes, it may be impractical to implant and process signals from the large number of electrodes typically used for dense electrode arrays and intracranial monitoring, as this would require too much computing power for an ambulatory system. Consequently, it would be desirable to reduce the number of monitored electrodes by determining the most important electrodes from the intracranial array or scalp array to determine the placement of a smaller number of permanently implanted electrodes. As can be appreciated, such a process would be required for each and every patient, and it is likely that the electrodes will be placed in different locations for each patient.

The present invention relates generally to electrode array patterns, placements of electrode arrays and methods of monitoring brain activity. More specifically, the present invention relates to predetermined electrode array patterns for monitoring brain activity signals for monitoring a patient's propensity for seizures.

One aspect of the invention provides a method of monitoring brain activity signals in a patient. In some embodiments, the method includes the steps of identifying a lobe or lobes of the brain in which the patient's seizures originate; based on the identified lobe or lobes of the brain, selecting an electrode array from a group of predetermined dispersed electrode array patterns; implanting the electrode array within the patient's cranium in contact with the identified lobe or lobes of the brain; and coupling the electrodes to an seizure advisory system. In some embodiments, the identification of the lobe or lobes of the brain does not include localization of a seizure focus. In some embodiments, the implanting of the electrode array includes implanting the electrode array to cover an area on the cortical surface which cannot be circumscribed by a circle having a radius of 4 cm. Additionally, the electrode array may include furcated electrode substrates.

In some embodiments, where the lobe or lobes of the brain include the frontal and temporal lobe, the step of implanting the electrode array includes the steps of implanting a first electrode array parallel to an interhemispheric fissure in the patient; implanting a second electrode array spanning between a precentral sulcus to an anterior edge of a temporal pole; implanting a third electrode array spanning between the precentral sulcus to the temporal pole; and implanting a fourth electrode array spanning between the precentral sulcus to a posterior portion of a Sylvian Fissure. The method may further include the step of surgically exposing part of a skull and creating a single opening approximately 1 cm anterior to the precentral sulcus and 1 cm lateral to the interhemispheric fissure and implanting the electrode arrays through the opening. In some embodiments, each electrode array may include 4 separate electrodes which are linearly spaced 20 mm apart from adjacent electrodes of the same array.

In some embodiments, where the lobe or lobes of the brain include the temporal lobe the step of implanting the electrode array includes the steps of implanting a first electrode array spanning from a middle temporal gyrus in line with a spinal cord to a temporal pole; implanting a second electrode array is inserted mesially from the middle temporal gyms in line with the spinal cord and wrapping under the temporal lobe; implanting a third electrode array spanning from the middle temporal gyms in line with the spinal cord posteriorly along the middle temporal gyrus; and implanting a fourth electrode array spanning from the middle temporal gyrus in line with the spinal into the temporal lobe both anteriorly and superiorly, passing approximately perpendicular to a Sylvian fissure. The method may further include the step of surgically exposing part of the skull, creating a single opening over the middle temporal gyrus in line with the spinal cord and implanting the electrode arrays through the opening. In some embodiments, the first, second and third electrode arrays each include four separate electrodes which are linearly spaced 10 mm apart from adjacent electrodes of the same array. The fourth electrode array may include four separate electrodes which are linearly spaced 20 mm apart from adjacent electrodes of the same array.

In some embodiments, where the lobe or lobes of the brain include the temporal and parietal lobes. In such embodiments, the step of implanting the electrode array includes the steps of implanting a first electrode array spanning between a precentral sulcus to an anterior edge of a temporal pole; implanting a second electrode array spanning between the precentral sulcus the temporal pole; implanting a third electrode array spanning between the precentral sulcus to a posterior portion of the Sylvian Fissure; and implanting a fourth electrode array spanning from the precentral sulcus posteriorly, approximately parallel to an interhemispheric fissure, projecting towards an occipital lobe. The method may further include the step of surgically exposing part of the skull and creating a single opening approximately 1 cm anterior to the precentral sulcus and 1 cm lateral to the interhemispheric fissure and implanting the electrode arrays through the opening. In some embodiments, each electrode array may include four separate electrodes which are linearly spaced 20 mm apart from adjacent electrodes of the same array.

Another aspect of the invention provides an EEG electrode array. In some embodiments, the EEG electrode array includes a substrate and a group of no more than 32 electrodes disposed on the substrate, configured to be inserted through a single opening in a patient's cranium and dispersed on a cortical surface of the brain radially away from the opening. In some embodiments, the electrodes are dispersed in a radial pattern larger than a circle having a radius of 4 cm. In some embodiments, the EEG electrode array has 16 electrodes. In some embodiments, the electrode array will also have two lead bodies, each lead body including a proximal end configured to couple to an implantable seizure advisory system and a bifurcated distal region having a first and second branch. In such embodiments, each branch has an electrode array with four electrodes.

Another aspect of the invention provides a seizure advisory system. In some embodiments, the system includes an electrode array having fewer than 32 electrodes in a predetermined dispersed radial pattern. The electrode array is adapted to be implanted through a single opening in the skull (such as a burr hole) and deployed in the predetermined dispersed radial pattern to detect a brain activity signal. While much of the discussion describes use of a burr hole to access an intracranial space, other openings, such as a craniotomy, may be used without departing from the invention. In some embodiments, the electrode array may be placed on the exterior of the skull beneath one or more layers of the scalp. The system also includes a communication assembly adapted to be implanted in the patient in communication with the electrode array. The communication assembly is configured to sample the brain activity signal with the electrode array. The system also includes an external assembly adapted to be positioned outside the patient's body, to receive a data signal from the communication assembly and to indicate a subject's susceptibility to a seizure. In some embodiments, the electrode array may include one or more depth electrodes, such as those typically used in deep brain stimulation. In some embodiments, the electrode array has no depth electrodes. In some embodiments, the spacing between adjacent electrodes within the same array is between 10 and 20 mm.

Another aspect of the invention provides a method for positioning electrodes for monitoring brain activity signals in a patient. In some embodiments, the method includes the steps of identifying a lobe or lobes of the patient's brain in which the patient's seizures originate; positioning 32 or fewer electrodes in a dispersed pattern on a surface of the brain such that the dispersal pattern cannot be circumscribed by a circle having a radius of 4 cm; and coupling the plurality of electrode arrays to a seizure advisory system. In some embodiments, the step of identifying a lobe or lobes of the brain in which the patient's seizures originate does not include localization of a seizure focus. In some embodiments, the step of identifying a lobe or lobes of the brain in which the patient's seizures originate includes the steps of localizing a seizure focus within the lobe or lobes of the brain, and the step of positioning the plurality of electrodes includes the step of ensuring that at least one of the electrodes is in proximity to the seizure focus. In some embodiments, the step of positioning the electrodes includes the step of inserting all of the electrodes through a single opening in the skull (such as a burr hole). In some embodiments, the step of positioning the electrodes further includes the step of positioning the electrodes on dura mater of the brain and/or positioning the electrodes underneath the dura mater and on a cortical surface of the patient's brain. In some embodiments, the step of positioning the electrodes includes positioning two or more substrates supporting the plurality of electrodes in a dispersed pattern on the brain. In some embodiments, the substrates are coupled to a common lead body adapted to connect to the seizure advisory system. In some embodiments, a first electrode and a second electrode of the plurality of electrodes are spaced greater than 8 cm apart.

In some embodiments, the method further includes the step of processing signals from at least one of the electrodes to estimate the patient's susceptibility for a seizure. In some embodiments, the method further includes the step of wirelessly transmitting a signal from the seizure advisory system to an external device. In some embodiments, at least a portion of the seizure advisory system is implanted within the patient's body.

For a further understanding of the nature and advantages of the present invention, reference should be made to the following description taken in conjunction with the accompanying drawings.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A to 1D illustrate electrode arrays that have a variety of different combinations of electrode contact sizes and electrode contact spacings.

FIG. 2 is a sagittal view of the first embodiment of a predetermined unilateral frontal and temporal lobe electrode array pattern for patients having a seizure focus regionalized in the frontal and temporal lobe region.

FIG. 3 is a sagittal view of the second embodiment of a predetermined unilateral temporal lobe electrode array pattern for patients having a seizure focus regionalized in the temporal lobe region.

FIG. 4. is a sagittal view of the third embodiment of a predetermined unilateral parietal and temporal lobe electrode array pattern for patients having a seizure focus regionalized in the parietal and temporal lobe region.

FIGS. 5A and 5B are sagittal views of an alternative embodiment of a predetermined bilateral electrode array pattern for monitoring a patient's brain activity that includes a lateral temporal lobe and medial temporal lobe array.

FIG. 5C is an axial view of the electrode array pattern shown in FIGS. 5A and 5B.

FIGS. 6A and 6B are sagittal views of an alternative embodiment of a predetermined bilateral electrode array pattern for monitoring a patient's brain activity that includes a medial temporal lobe and anterior polar temporal lobe array.

FIG. 6C is an axial view of the electrode array pattern shown in FIGS. 6A and 6B.

FIGS. 7A and 7B are sagittal views of an alternative embodiment of a predetermined bilateral electrode array pattern for monitoring a patient's brain activity that includes a lateral temporal lobe and anterior polar temporal lobe array.

FIG. 7C is an axial view of the electrode array pattern shown in FIGS. 7A and 7B.

FIGS. 8A and 8B are sagittal views of an alternative embodiment of a predetermined bilateral electrode array pattern for monitoring a patient's brain activity that includes a lateral temporal lobe, medial temporal lobe, and anterior polar temporal lobe array.

FIG. 8C is an axial view of the electrode array pattern shown in FIGS. 8A and 8B.

FIGS. 9A and 9B are sagittal views of an alternative embodiment of a predetermined bilateral electrode array pattern for monitoring a patient's brain activity that includes a lateral temporal lobe and medial temporal lobe array.

FIG. 9C is an axial view of the electrode array pattern shown in FIGS. 9A and 9B.

FIGS. 10A and 10B are sagittal views of an alternative embodiment of a predetermined bilateral electrode array pattern for monitoring a patient's brain activity that includes a medial temporal lobe and anterior polar temporal lobe array.

FIG. 10C is an axial view of the electrode array pattern shown in FIGS. 10A and 10B.

FIGS. 11A and 11B are sagittal views of an alternative embodiment of a predetermined bilateral electrode array pattern for monitoring a patient's brain activity that includes a lateral temporal lobe and anterior polar temporal lobe array.

FIG. 11C is an axial view of the electrode array pattern shown in FIGS. 11A and 11B.

FIGS. 12A and 12B are sagittal views of an alternative embodiment of a predetermined bilateral electrode array pattern for monitoring a patient's brain activity that includes a lateral temporal lobe, medial temporal lobe, and anterior polar temporal lobe array.

FIG. 12C is an axial view of the electrode array pattern shown in FIGS. 12A and 12B.

FIG. 13 illustrates a set of bifurcated electrode arrays having a single lead.

FIG. 14 illustrates multiple electrode arrays including integrally formed electrode arrays corresponding to the electrode array patterns of FIGS. 5A-12C.

FIG. 15 illustrates one example of a patient system with which the electrode array pattern may be used.

FIG. 16 illustrates a kit embodied by the present invention.

FIG. 17 illustrates an example of a method embodied by the present invention.

### DETAILED DESCRIPTION OF THE INVENTION

Certain specific details are set forth in the following description and figures to provide an understanding of various embodiments of the invention. Certain well-known details, associated electronics and devices are not set forth in the following disclosure to avoid unnecessarily obscuring the various embodiments of the invention. Further, those of ordinary skill in the relevant art will understand that they can practice other embodiments of the invention without one or more of the details described below. Finally, while various processes are described with reference to steps and sequences in the following disclosure, the description is for providing a clear implementation of particular embodiments of the invention, and the steps and sequences of steps should not be taken as required to practice this invention.

The term “condition” is used herein to generally refer to the patient's underlying disease or disorder—such as epilepsy, depression, Parkinson's disease, headache disorder, etc. The term “state” is used herein to generally refer to calculation results or indices that are reflective of a categorical approximation of a point (or group of points) along a single or multi-variable state space continuum. The estimation of the patient's state does not necessarily constitute a complete or comprehensive accounting of the patient's total situation. As used in the context of the present invention, state typically refers to the patient's state within their neurological condition. For example, for a patient suffering from an epilepsy condition, at any point in time the patient may be in a different states along the continuum, such as:

i) An ictal state (which within the scope of epilepsy, refers to seizure activity).

ii) An inter-ictal state is sometimes termed the “normal” neurological state and represents the state in between seizures.

iii) A post-ictal state which is the neurological state immediately following a seizure or ictal state.

iv) A pro-ictal state represents a state of high susceptibility for seizure; in other words, a seizure can happen at any time. Some researchers have proposed that seizures develop minutes to hours before the clinical onset of the seizure. These researchers therefore classify a pre-ictal condition as the beginning of the ictal or seizure event which begins with a cascade of events. Under this definition, a seizure is imminent and will occur if a pre-ictal condition is observed. Others believe that a pre-ictal condition represents a state which only has a high susceptibility for a seizure and does not always lead to a seizure and that seizures occur either due to chance (e.g., noise) or via a triggering event during this high susceptibility time period. For clarity, the term “pro-ictal” is used herein to describe a general state or condition during which the patient has a high susceptibility for seizure. Accordingly, the pre-ictal state as used in either definition above would be considered to be a pro-ictal state.

v) A contra-ictal state in which the subject is highly unlikely to transition to the ictal state within a specified time period. This contra-ictal condition can be considered to be a subset of the inter-ictal class or it can be considered to be a completely new neurological classification. While it is beneficial to the subject to know if the subject is in an inter-ictal condition, being in an inter-ictal condition does not necessarily inform the subject that they will not quickly transition from the inter-ictal condition to an ictal condition. Being able to inform a subject that they are in a contra-ictal state can allow the subject to engage in normal daily activities, such as driving a car, or walking down a set of stairs, without fearing that they will have a seizure or without fearing that they may quickly transition into a pro-ictal state.

The estimation and characterization of state may be based on one or more patient dependent parameters from the a portion of the patient's body, such as electrical signals from the brain, including but not limited to electroencephalogram signals and electrocorticogram signals (“ECoG”) or intracranial EEG (referred to herein collectively as “EEG”), brain temperature, blood flow in the brain, concentration of anti-epileptic drugs (referred to as “AEDs”) in the brain or blood, etc. While parameters that are extracted from brain-based signals are preferred, the present invention may also extract parameters from other portions of the body, such as the heart rate, respiratory rate, chemical concentrations, etc.

An “event” is used herein to refer to a specific event in the patient's condition. Examples of such events include transition from one state to another state, e.g., an electrographic onset of seizure, end of seizure, or the like. For conditions other than epilepsy, the event could be an onset of a migraine headache, a tremor, or the like.

The occurrence of a seizure may be referred to as a number of different things. For example, when a seizure occurs, the patient is considered to have exited a pro-ictal state and has transitioned into an ictal state. However, the electrographic onset of the seizure (one event) and/or the clinical onset of the seizure (another event) have also occurred during the transition of states.

As used herein, a patient's “propensity” for a seizure is a measure of the likelihood of transitioning into the ictal state. The patient's propensity for seizure may be estimated by determining which state the patient is currently in. As noted above, the patient is deemed to have an increased propensity for transitioning into the ictal state (e.g., have a seizure) when the patient is determined to be in a pro-ictal state. Likewise, the patient may be deemed to have a low propensity for transitioning into the ictal state for a period of time when it is determined that the patient is in a contra-ictal state.

In one aspect, the present invention provides for predetermined dispersed electrode array patterns that are useful for monitoring activity in specific brain regions for a broad array of patients having epilepsy. Instead of requiring extensive localization techniques to attempt to pin-point the epileptic focus to determine the appropriate positions of the electrode arrays, the present invention may be used as substantially universal electrode placement configurations to sample brain activity signals for the monitoring of neurological and psychiatric conditions. Such universal electrode placement configurations or predetermined electrode array patterns do not require pin-point localization of the seizure focus (e.g., 1 cm resolution). Rather, the configuration is based upon the regionality or general location of the seizure focus. Such seizure focus regions can be classified as i) the frontal and temporal lobe region, ii) temporal lobe region and iii) temporal and parietal lobe region. In various embodiments, monitoring or imaging may or may not be used, and the patient may undergo non-invasive and/or invasive monitoring to assess their condition and identify a seizure focus, if desired.

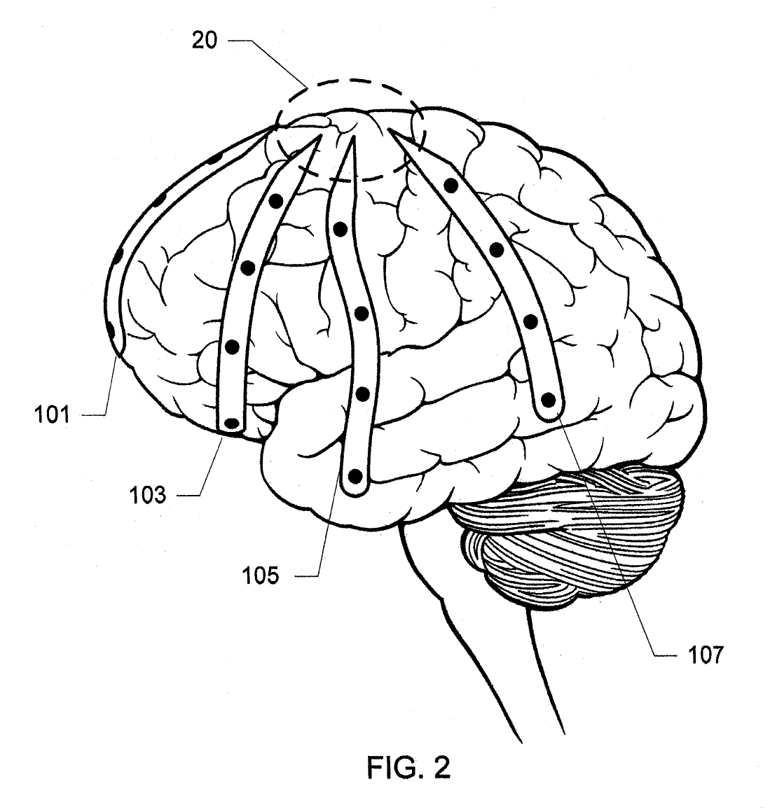

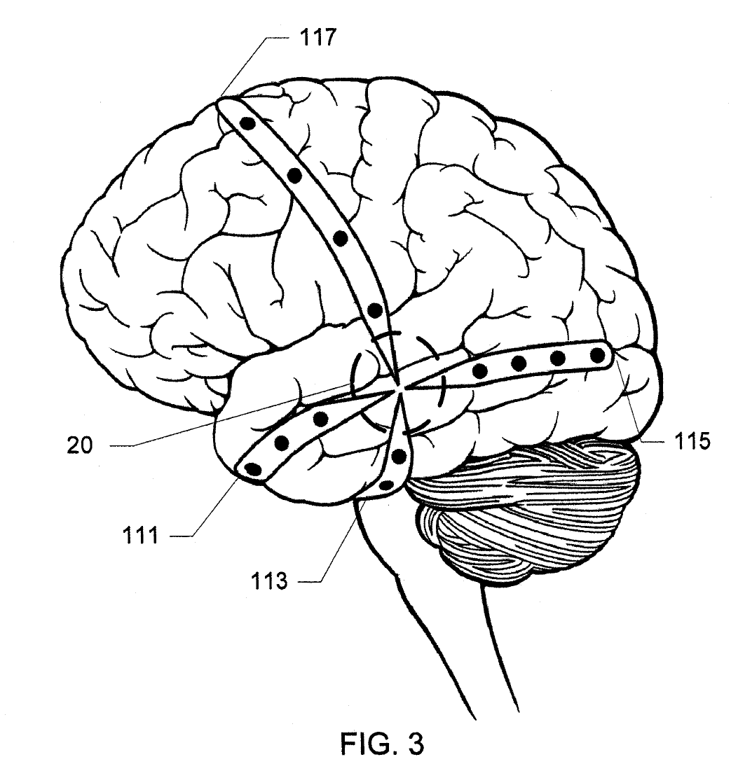

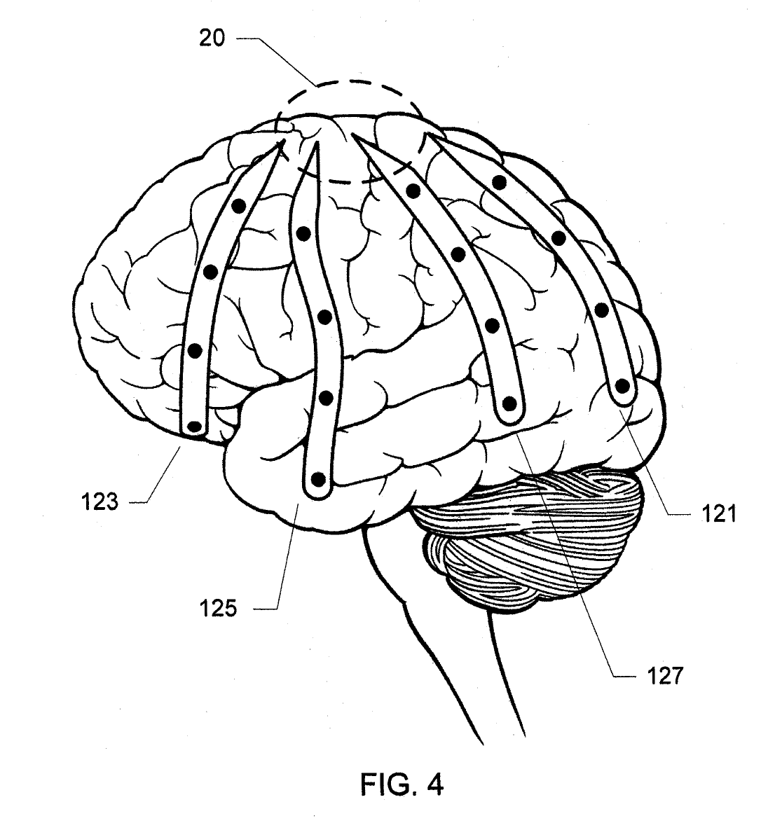

Conventional dense grid electrode arrays typically have 64 or more electrodes spaced 10 mm apart (yielding an electrode density of 1 electrode per cm²). These dense grid arrays are typically used in the extensive localization techniques described above, and can be highly invasive. Upon locating the seizure focus, the electrodes located right at the focus are activated, or additional electrodes will be implanted at the epileptic focus, to detect brain activity. In contrast, the predetermined electrode array patterns in accordance with embodiments of this invention are placed over an entire region of the patient's brain. Thus, when implanted in a patient to monitor and assess the patient's brain state, the predetermined electrode arrays purposefully have electrodes that lie outside of the seizure focus and that can detect a brain activity signal from areas of the brain outside of the seizure focus. The predetermined electrode patterns have 32 or fewer electrodes which are deployed in configurations that are illustrated in FIG. 2 (frontotemporal lobe region), FIG. 3 (temporal lobe region) and FIG. 4 (temporal and parietal lobe regions) and extend over brain surface areas corresponding to approximately 87 cm², 50 cm² and 87 cm², respectively. In embodiments of the invention using a total of 16 electrodes, the dispersed electrode patterns may have an electrode density of 0.33 or 0.20 electrodes per cm².

The predetermined electrode array patterns of the present invention may be useful for implantable, ambulatory monitoring of the patient's brain activity, as well as in-hospital monitoring in units such as an epilepsy monitoring unit (EMU). As will be described in more detail below, in preferred embodiments the signals sampled from the patient's brain with the predetermined electrode array pattern may be processed to estimate the patient's propensity for transitioning into an ictal state (sometimes referred to herein as “seizure advisory”).

While the remaining discussion focuses on monitoring epileptic patients, the present invention may also be applicable to monitoring other neurological or psychiatric conditions. For example, the present invention may also be applicable to monitoring and management of sleep apnea and other sleep disorders, Parkinson's disease, Huntington's disease, essential tremor, Alzheimer's disease, migraine headaches, depression, rigidity, hemiballism, choreoathetosis, dystonia, akinesia, bradykinesia, hyperkinesia, anxiety, phobia, schizophrenia, multiple personality disorder, eating disorders, bipolar spectrum disorders, or other psychiatric or neurological conditions. As can be appreciated, features extracted from the sampled signals and used by algorithms or other analysis systems will be specific to the underlying disorder that is being managed. While certain features may be relevant to epilepsy, such features may or may not be relevant to the state measurement for other conditions.

The electrode arrays in accordance with embodiments of the present invention may be implanted into the brain, subdurally, epidurally, partially or fully in the skull, or between the skull and one or more layers of the patient's scalp. In some embodiments, the electrode array is configured to be inserted through a single opening in a skull of a patient and to be dispersed on a surface of the brain. The surface is preferably the cortical surface of the brain, but may alternatively be any other suitable surface or layer of the brain, such as the dura mater. The electrodes are preferably dispersed radially out from the single opening, but may alternatively be dispersed in any other suitable configuration. In some embodiments, the electrodes are dispersed over an area of the brain larger than a circle having a radius of 4 cm. In some embodiments, the electrode array patterns may include 32 or fewer electrodes, and in some embodiments the electrode array patterns may include 16 or fewer electrodes.

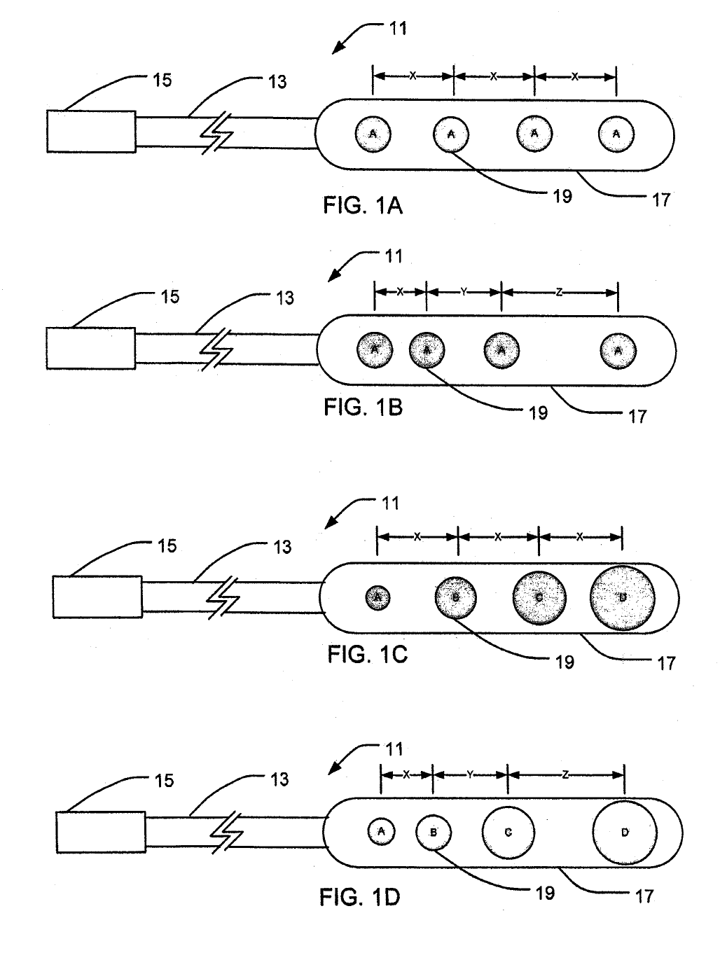

The electrode arrays 11 shown in FIGS. 1A-1D each include a plurality of electrodes 19 supported by lead bodies. The lead bodies have a proximal connector 15 configured to couple to a seizure advisory system (not shown) and a distal body 17 supporting the electrodes. A lead 13 connects the proximal connector 15 to the distal body 17 and electrodes 19. In one embodiment, the distal bodies 17 are in the form of a substantially flat, flexible substrate that is configured for placement on a cortical surface of the patient's brain. The electrode contacts 19 are used to sample brain activity signals from the patient's brain.

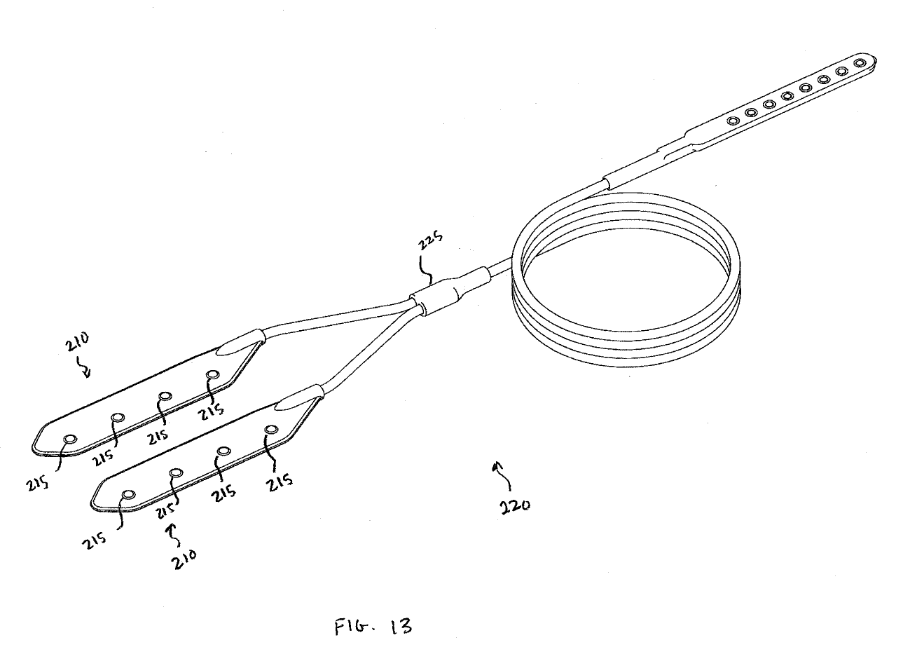

In one variation, as shown in FIG. 13, the plurality of electrodes include furcated electrode distal bodies, having two or more distal bodies each containing a plurality of electrodes and coupled to a common lead body. For example, the lead bodies may include a bifurcated distal region having a first branch and a second branch. Each branch may include an electrode array comprising a number of electrodes. In this variation, each distal body includes four electrodes, but may alternatively include any other suitable number of electrodes.

The electrode array patterns may include multiple electrode arrays disposed in a specific configuration over the patient's brain, such as on a cortical surface. As can be appreciated, the distal body of embodiments that may be implanted deep within brain tissue will typically have a more elongate, rigid distal body than the flexible substrates used for placement on the cortical surface.

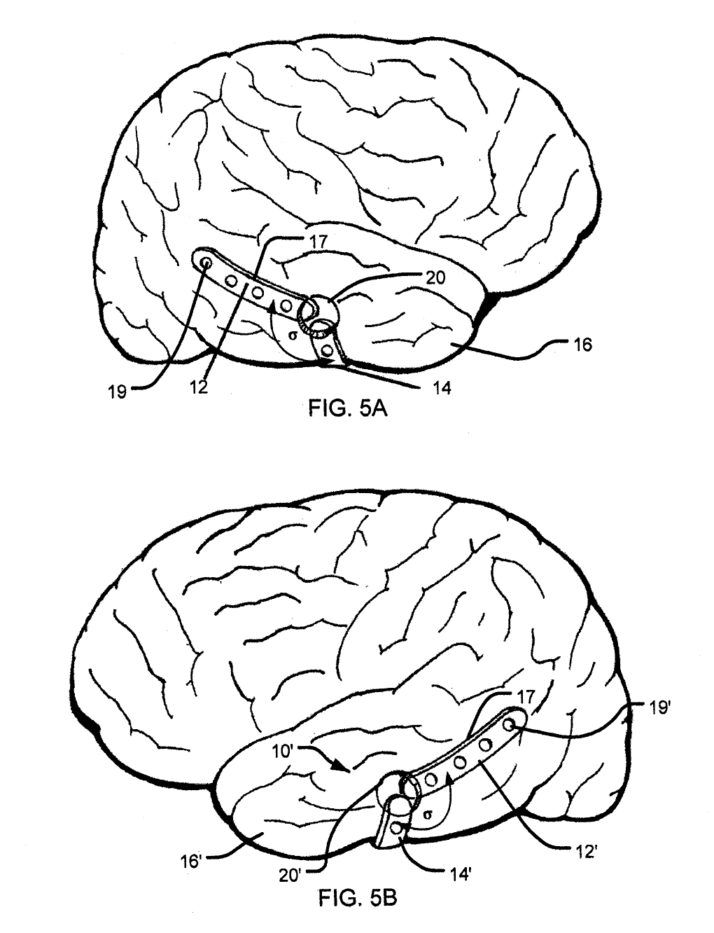

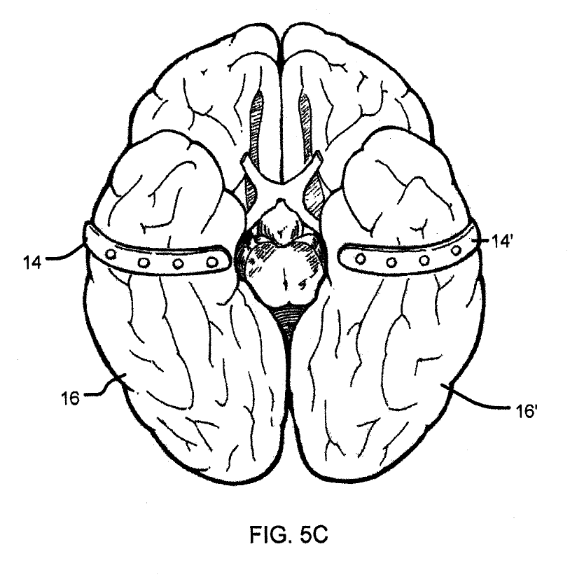

FIGS. 2-12C illustrate a number of different predetermined electrode array patterns that are embodied by the present invention. FIGS. 2-4 illustrate electrode array patterns which are unilaterally implanted (e.g., on one side of the temporal lobe), whereas FIGS. 5A-5C, 6A-6C, 7A-7C, 8A-8C, 9A-9C, 10A-10C, 11A-11C, and 12A-12C illustrate electrode array patterns implanted bilaterally over the patient's temporal lobes.

As shown in FIGS. 2-12, the electrode arrays are typically implanted subdurally into the patient through a single opening in the skull, such as a burr hole 20 or craniotomy. The size of the dura mater penetrations should be minimized, yet large enough to permit proper placement of the electrode. Of course, for placement of the additional distal electrode(s) (not shown) or bilateral electrodes, additional burr holes may be needed. The electrode arrays are typically sized and shaped so as to be inserted into the patient's intracranial cavity and placed over the frontal, temporal or parietal lobe through the one or more burr holes, in which the proximal ends of the distal bodies are positioned adjacent the burr hole. The lead body (e.g., element 13 in FIGS. 1A-1D) extending from the electrode substrate may then be threaded through the burr hole 20 and tunneled between the scalp and cranium and down the neck to a subclavicularly placed implanted device (described below). The implanted device need not be limited to the subclavicular area; it could also be located within the cranium, abdomen or other areas within the body. It may also be possible to have one or more of the electrode arrays in the form of depth electrodes.

The predetermined electrode array pattern may be made of multiple electrode arrays. Also, as shown in FIGS. 1A-D, the distal body 17 and electrodes 19 of the electrode array 11 may have a variety of different dimensions. For the case when there are multiple electrode arrays making up the electrode array pattern, each array may have the same dimensions as each other or different dimensions from each other. For example, the distal bodies of the electrode arrays will have a length between about 40 mm and about 80 mm, a width between about 5 mm and about 15 mm, and a thickness between about 0.02 inches and about 0.10 inches.

A distal body 17 of the electrode array 11 and the electrode array contacts 19 may be made from any of the commonly used bio-compatible materials used for permanently or temporarily implanted electrode arrays. Some materials include, but are not limited to, silicone distal body material, platinum iridium electrodes, stainless steel electrodes, MP35N alloy electrodes, or the like.

FIGS. 1A-1D illustrate a variety of different electrode contact 19 sizes (A, B, C, D) and electrode spacings (X, Y, Z). The center-to-center spacing (generically referred to herein as “spacing”) between the adjacent electrode contacts 19 on the electrode array 11 may be the same (e.g., X in FIGS. 1A, 1C) or may vary (e.g., X, Y, Z in FIGS. 1B, 1D). The variance in the spacing between the contacts 19 may vary in an exponential, linear, or non-linear manner. For example, the burr hole may be created and the electrode array 11 may be inserted into the patient's skull, with a proximal end of the distal body 17 of electrode array adjacent the burr hole. The electrode spacing between the adjacent contacts 19 may increase (or decrease) linearly, non-linearly, exponentially, etc., as the electrode contacts 19 become more distal to the burr hole. One embodiment utilizes electrode spacing of greater than 8 mm, such as 10 mm or 20 mm. Additionally, that embodiment utilizes an electrode contact area of 4.9 mm². Additionally, as shown there may be four electrodes per substrate; however, this number may increase up to 32 electrodes per substrate.

The spacing between the adjacent electrode contacts may depend on the length of the distal body of the electrode array, the size of the electrode contacts, the number of electrode contact on each array, the type of electrode array, the position of the electrode array over the brain, the type of neurological disorder being monitored, etc. The electrode spacing may be, for example, between 5 mm and about 50 mm, and, more specifically, between about 10 mm and about 20 mm.

The electrode contacts may have a round, elliptical, or oval shape, or may have any other desired shape. The electrode contacts 19 themselves may have, for example, an exposed surface area between about 0.5 mm² and about 20 square mm², and preferably between about 2 mm² and about 10 mm².

The electrode contacts on each of the electrode arrays may have the same dimensions as the other electrode contacts on the array, or they may have different dimensions from the other electrode contacts on the array. For example, similar to the spacing between adjacent electrode contacts, in some embodiments, the size of the contacts may change from a distal end of the array to the proximal end of the array from the burr hole. Each of the arrays in the electrode array pattern may have the same size electrode contacts as the other arrays in the electrode array pattern (e.g., A-sized electrodes in FIGS. 1A, 1B) or they may have different electrode contact sizes from the other electrode arrays in the electrode array pattern (e.g., different sizes A, B, C, D in FIGS. 1C-1D). In one embodiment, the electrode contact that is most proximal to the burr hole may be larger than the electrode contact 19 that is most distal from the burr hole. Alternatively, the electrode contact that is most distal from the burr hole may be larger than the electrode contact that is most proximal from the burr hole. The size of the electrode contacts from proximal to distal (or distal to proximal) may vary linearly, non-linearly, exponentially, etc.

In some configurations, it may be desirable to mark the electrode arrays with an indication of their placement over the temporal, frontal or parietal lobes and there may be markings which indicate which end of the array is to be distal and/or proximal to the burr hole. The markings may be text, numbers, color, or the like.

The electrode arrays in the electrode array patterns may have any number of individual electrode contacts as desired. The electrode arrays may have the same number of electrode contacts or different number of electrode contacts as the other electrode arrays in the electrode array patterns. Typically, each electrode array will include between two electrode contacts and about 16 electrode contacts. In most embodiments in which the electrode arrays are implanted in the patient and a processing assembly is implanted in the body of the patient, the total number of electrode contacts in the electrode array pattern is between about two electrode contacts and about 16 electrode contacts, and such as between about four electrode contacts and about eight electrode contacts. Of course, for certain neurological conditions, it may be possible to process signals from as few as one electrode, but for other neurological conditions it may be desirable to process signals from 32 channels or more.

FIGS. 2-12C show sagittal and axial views of various embodiments of predetermined electrode array patterns that are encompassed by the present invention. FIGS. 2-4 represent predetermined electrode array patterns for the following seizure focus locations that have been regionalized to the following regions of the brain: frontal, frontal-temporal, temporal, bilateral temporal, parietal and parietal-temporal lobes, respectively. Table 1.0 illustrates predetermined electrode array patterns as a function of the seizure region location. Note: subjects with bilateral temporal onset will typically be lateralized to the hemisphere that generates the most frequent, stereotypical seizure (if possible).

TABLE 1.0
Electrode placement scheme selection

| Seizure focus location | Electrode placement scheme |
|---|---|
| Frontal | Frontotemporal (as shown in FIG. 2) |
| Frontotemporal | |
| Temporal | Temporal (as shown in FIG. 3) |
| Bilateral temporal | |
| Parietal | Parietal-temporal (as shown in FIG. 4) |
| Parietal-temporal | |

The predetermined frontotemporal electrode array pattern 10 which is used for seizure foci regionalized either in the frontal or frontotemporal lobes is illustrated in FIG. 2 and typically includes four separate electrode arrays 101, 103, 105 and 107 inserted via a single burr hole 20 or craniotomy placed approximately 1 cm anterior to the precentral sulcus and 1 cm lateral to the interhemispheric fissure. Each of the four electrode arrays may include four individual electrodes, such that the entire array pattern includes sixteen total electrodes. The individual electrodes are spaced greater than 10-20 mm apart from the adjacent electrodes on the same array. The first electrode array 101 is inserted anteriorly parallel to the interhemispheric fissure and may wrap around the frontal pole. The second electrode array 103 is inserted laterally and anteriorly toward the anterior edge of the temporal pole and may wrap under the frontal lobe, preferably spanning between a precentral sulcus to an anterior edge of a temporal pole. The third electrode array 105 is inserted laterally toward the temporal pole and may wrap under the temporal lobe, preferably spanning between the precentral sulcus to the temporal pole. The fourth electrode array 107 is inserted laterally and posteriorly toward the posterior portion of the Sylvian fissure, preferably spanning between the precentral sulcus to a posterior portion of a Sylvian Fissure. If additional localization information is available regarding the seizure focus beyond that of regionalization, the position of the predetermined electrode array pattern may be further optimized during implantation in order to ensure that at least one of the electrodes is in proximity to the seizure focus.

This procedure is applicable to the additional array patterns presented from FIG. 2 through FIG. 12C. The predetermined temporal electrode array pattern used for seizure foci regionalized either in the temporal lobe or bilateral temporal lobes is illustrated in FIG. 3 and typically includes four separate electrode arrays 111, 113, 115 and 117 inserted via a single burr hole 20 or craniotomy placed over the middle temporal gyrus in line with the spinal cord. Each of the four electrode arrays may include four individual electrodes such that the entire array pattern includes sixteen total electrodes. The individual electrodes on the first, second, and third array are may be spaced 10 mm apart from the adjacent electrodes on the same array. The individual electrodes of the fourth array are may be spaced 20 mm apart from the adjacent electrodes on the same array. The first electrode array 111 is inserted anteriorly toward the temporal pole, preferably spanning from a middle temporal gyrus in line with a spinal cord to a temporal pole. The second electrode array 113 is inserted mesially from the middle temporal gyrus in line with the spinal cord and wrapping under the temporal lobe. The third electrode array 115 is inserted posteriorly along the middle temporal gyrus, preferably spanning from the middle temporal gyrus in line with the spinal cord posteriorly along the middle temporal gyrus. The fourth electrode array 117 is inserted anteriorly and superiorly, preferably spanning from the middle temporal gyrus in line with the spinal into the temporal lobe both anteriorly and superiorly, passing perpendicular to the Sylvian fissure.

The parietal-temporal electrode array pattern used for seizure foci regionalized either in the parietal lobe or near the parietal/temporal lobe boundary is illustrated in FIG. 4 and typically includes four separate electrode arrays 121, 123, 125 and 127 inserted via a single burr hole 20 or craniotomy placed approximately 1 cm anterior to the precentral sucus and 1 cm lateral to the interhemispheric fissure. On each of the four electrode arrays, the individual electrodes are may be spaced than 10-20 mm apart from the adjacent electrodes on the same array. The parietal-temporal electrode array pattern is substantially identical to the frontal-temporal array of FIG. 2, except the first electrode array 121 is inserted posteriorly, parallel to the interhemispheric fissure, projecting towards the occipital lobe. The second 123, third 125 and fourth 127 electrode arrays are placed identically to the frontal-temporal electrode array pattern.

FIGS. 5A-12C illustrate alternative embodiments that utilize bilateral implementation of electrode array pattern. As shown in FIGS. 5A and 5B, one embodiment of the electrode array pattern includes electrode arrays 12, 12′, 14, 14′. The illustrated electrode array pattern includes lateral temporal strip electrode arrays 12, 12′ and medial temporal strip electrode arrays 14, 14′ for monitoring brain activity signals from the patient's temporal lobes. Because of the relatively small size of the temporal lobe 16, 16′ (relative to the other lobes of the patient's brain), only a small number of electrode contacts 19, 19′ may be needed to adequately sample the brain activity signals from one or more temporal lobes 16, 16′ of the patient. In the illustrated example, the electrode arrays 12, 12′, 14, 14′ may be inserted through burr holes 20, 20′ that are created over each of the temporal lobes 16, 16′, and slipped beneath the dura mater (not shown). As shown in FIG. 5C, the medial temporal strip electrode array 14, 14′ extends from the burr hole and over a portion of the inferior cortical surface of the temporal lobe 16, 16′. As shown in FIG. 5C, for embodiments which have bilateral implantation, the medial temporal strip electrode arrays 14, 14′ may be parallel to each other and substantially aligned with each other over the temporal lobes 16, 16′. In alternative embodiments, the medial temporal strip electrode arrays 14, 14′ may be non-parallel with each other and/or misaligned with each other.

In the illustrated embodiment, the bilateral system illustrates sixteen total electrode contacts (and hence 16 channels of data—8 channels of data from each temporal lobe). As noted above, however, while 16 channels of data are shown, any number of electrode contacts may be used with the electrode array pattern of the present invention.

Referring again to FIGS. 5A and 5B, the lateral temporal strip electrode arrays 12, 12′ and the medial temporal strip electrode arrays 14, 14′ may be arranged so that proximal ends of the distal body of each of the strip electrode arrays are adjacent each other at or near the burr holes 20, 20′. Such electrode arrays 12, 12′, 14, 14′ define an angle between the longitudinal axes defined by the body of the electrode arrays. The angle, σ, may be between about 70 degrees and about 120 degrees.

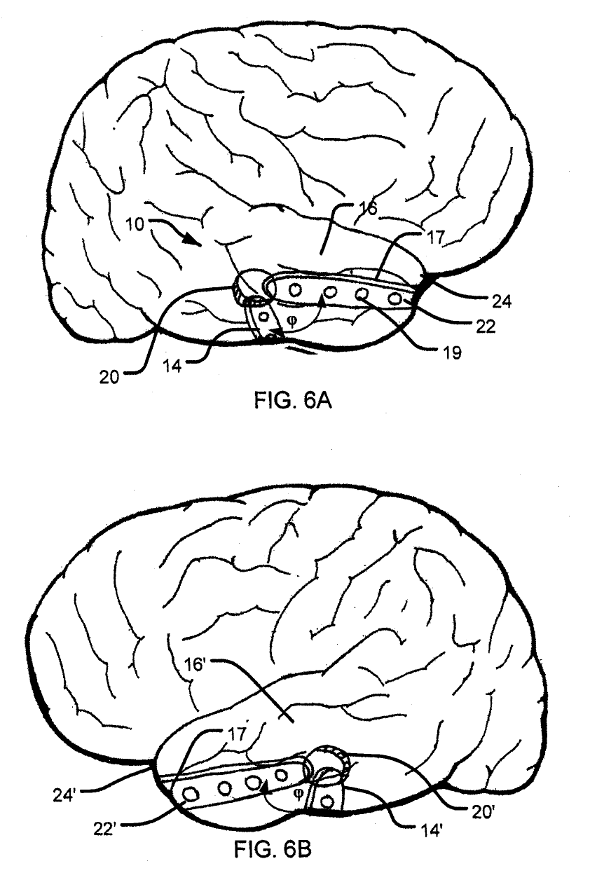

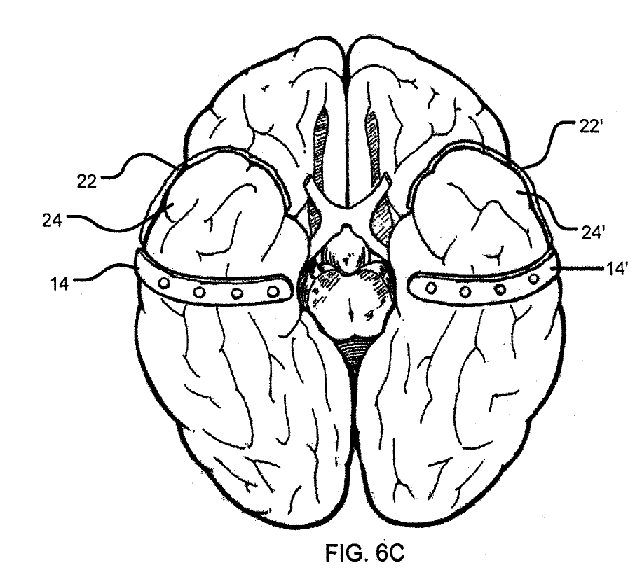

FIGS. 6A-6C illustrate another electrode array pattern which includes medial temporal strip electrode arrays 14, 14′ and anterior polar electrode arrays 22, 22′ that wrap around an anterior surfaces 24, 24′ of the temporal lobes 16, 16′. The medial temporal strip electrode arrays 14, 14′ and anterior polar strip electrode arrays 22, 22′ may be arranged so that proximal ends of each of the distal body 17 of the strip electrodes 14, 14′, 22, 22′ are adjacent each other at or near the burr holes 20, 20′. Such electrode arrays 14, 14′, 22, 22′ define an angle between the longitudinal axes defined by the longitudinal axes of the distal bodies 17 of the electrode arrays. The angle, φ, may be between about 70 degrees and about 120 degrees.

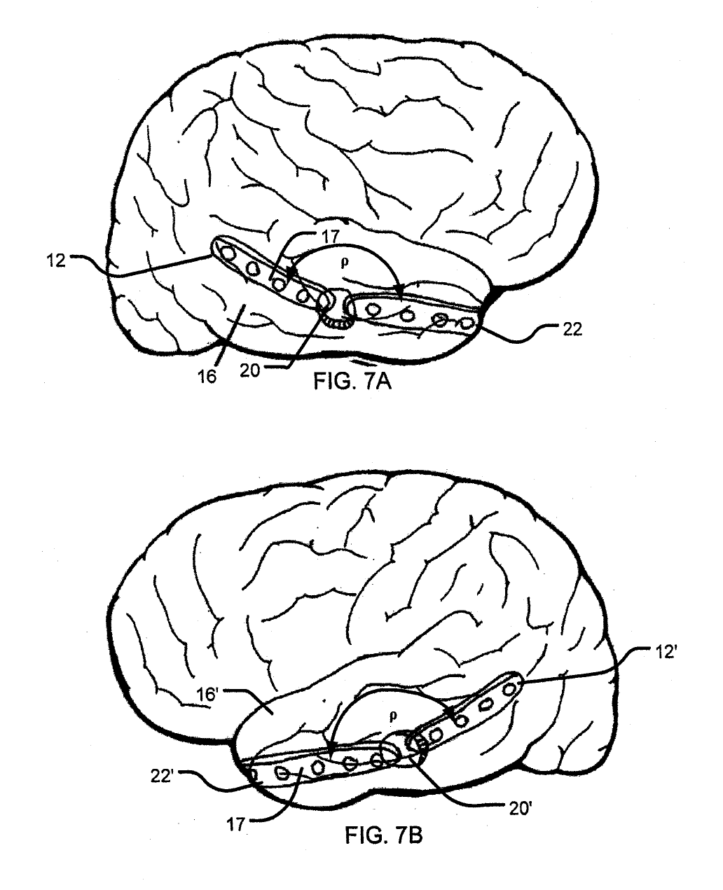

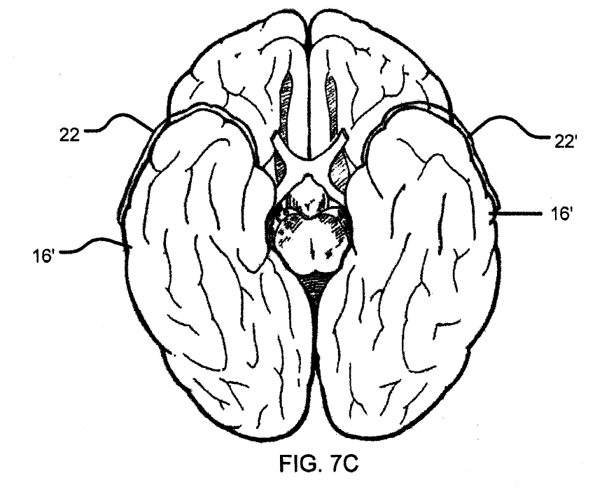

FIGS. 7A-7C illustrate another electrode array pattern which includes lateral temporal strip electrode arrays 12, 12′ that extend posteriorly along the temporal lobes from the burr holes 20, 20′ and anterior polar electrode arrays 22, 22′ that wrap around the anterior surfaces 24, 24′ of the temporal lobes 16, 16′.

Similar to the other embodiments, the lateral temporal strip electrode arrays 12, 12′ and anterior polar strip electrode arrays 22, 22′ may be arranged so that proximal ends of each of the distal bodies 17 of the strip electrodes 12, 12′, 22, 22′ are adjacent each other at or near the burr holes 20, 20′. Such electrode arrays 12, 12′, 22, 22′ define an angle between the longitudinal axes of the distal bodies of the electrode arrays. The angle, p, may be between about 150 degrees and about 210 degrees.

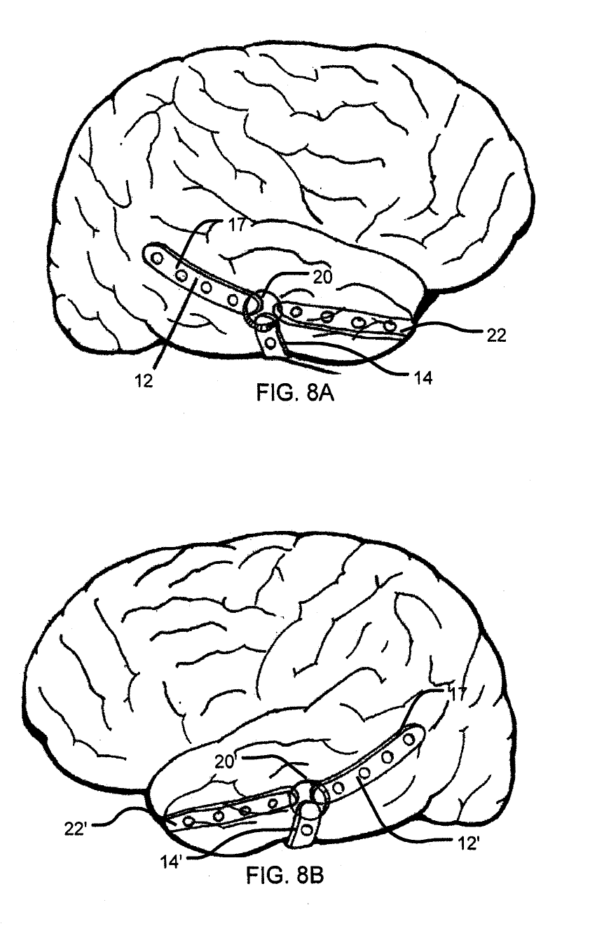

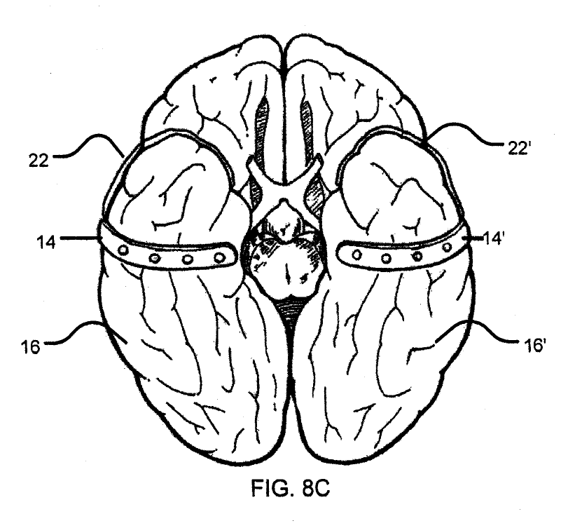

FIGS. 8A-8C illustrate another electrode array pattern, which includes lateral temporal strip electrode arrays 12, 12′, medial temporal strip electrode arrays 14, 14′ that extend posteriorly along the temporal lobe from the burr holes 20, 20′ and anterior polar electrode arrays 22, 22′ that wrap around the anterior surfaces 24, 24′ of the temporal lobes 16, 16′. The illustrated configuration provides 24 total channels of data (12 channels of data from each temporal lobe) and provides the most coverage of the temporal lobe, relative to the other embodiments described above. While FIGS. 8A-8C illustrate 12 channels that are monitored over each temporal lobe, it should be appreciated that any number of channels can be sampled. The relative orientations between each of the electrode arrays 12, 14, 22 will be similar to the embodiments described above.

The illustrated configuration provides the most complete coverage over the temporal lobe, but it also provides the largest number of channels that need to be processed. If desired, the systems which use the electrode array pattern of the present invention may be configured to sample and process signals from selected contacts from the electrode array pattern that are most predictive for that particular patient. Selection of the contacts (and reselection of the contacts) may be accomplished in vivo and may be performed through reprogramming by the physician.

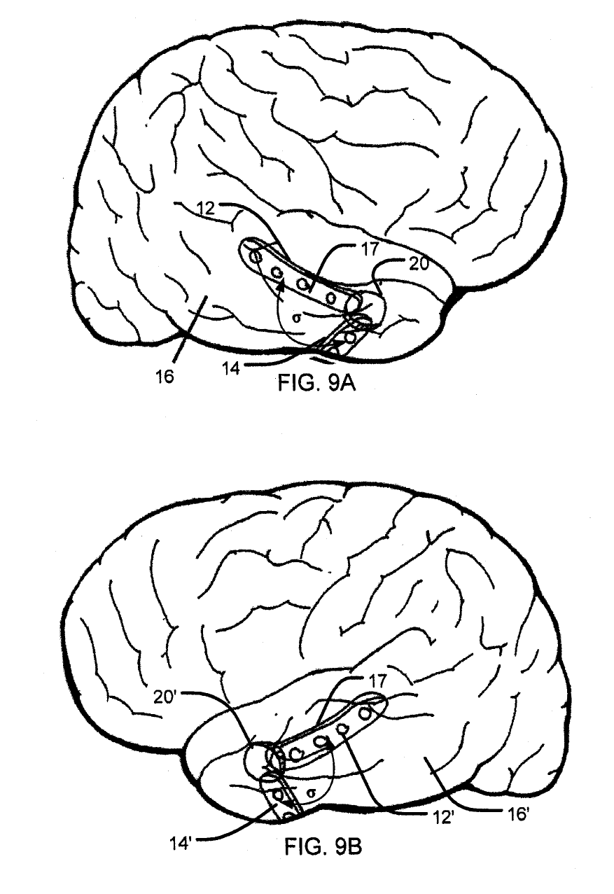

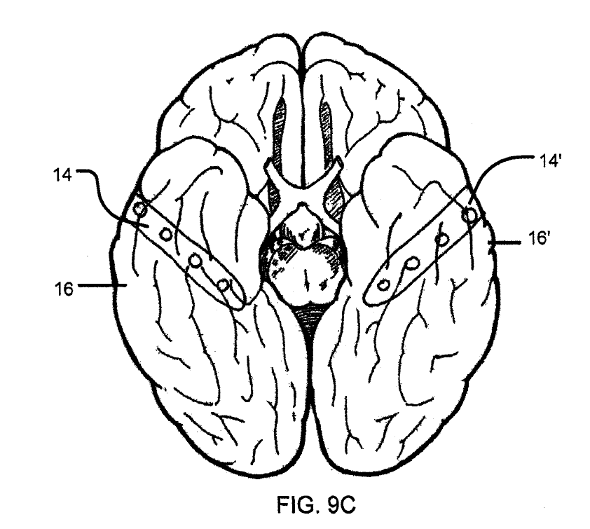

FIGS. 9A-12C illustrate electrode array embodiments that are similar to the embodiments illustrated in FIGS. 3A-8C, respectively. The primary difference between the different embodiments is the more anterior placement of the burr holes 20, 20′ in the skull and the relative orientations of the various electrode arrays in the pattern. The burr holes may be placed somewhat central to the desired electrode dispersion. There are also anatomical considerations, of course, controlling placement of the opening. Thus, while not shown, in other embodiments, the burr holes could be placed more superior and/or inferior from the placement shown in FIGS. 5A-8C.

For example, as shown in FIG. 9C, because the burr holes 20, 20′ are anterior of the burr holes of the previous embodiments, it may be desirable advance a distal tip of the medial temporal electrode array 14, 14′ more posteriorly so that distal ends of the distal bodies of the medial temporal electrode arrays 14, 14′ are posterior of proximal ends of medial temporal electrode arrays and the burr holes 20, 22. As such, as shown in FIG. 9C, the medial temporal electrode arrays 14, 14′ will no longer be substantially parallel with each other. Because of the different orientation between the two electrode arrays 12, 14, the angle σ between the lateral temporal electrode array and the medial temporal electrode array may differ from the others. In the embodiment illustrated in FIGS. 9A-9C, the angle σ may be between about 70 degrees and about 120 degrees.

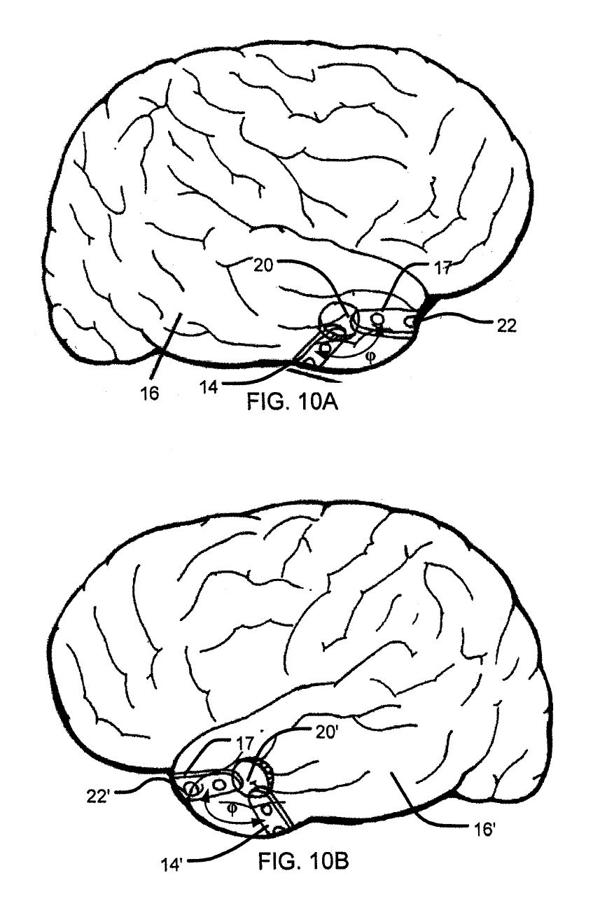

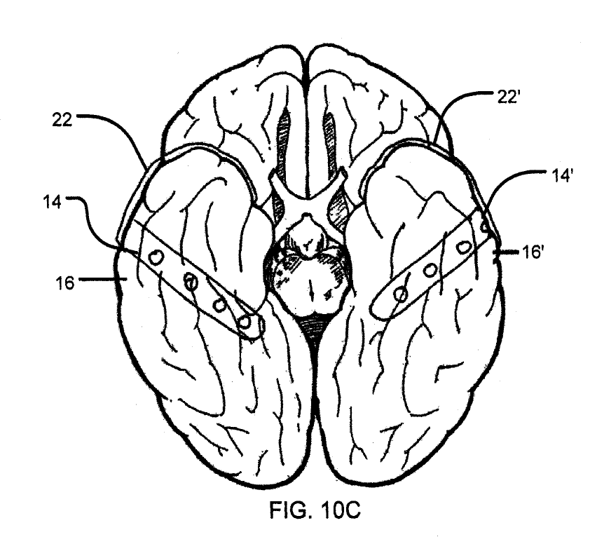

Similarly FIGS. 10A-10C illustrate medial temporal electrode arrays 14, 14′ and anterior polar electrode arrays 22, 22′ that are inserted into a patient with more anteriorly positioned burr holes 20, 20′. As shown in FIG. 10C, because the burr holes 20, 20′ are anterior of the burr holes of the embodiment shown in FIGS. 6A-6C, it may be desirable advance distal ends of the distal bodies 17 of the medial temporal electrode arrays 14, 14′ more posteriorly so that the distal ends of the distal bodies 17 of the medial temporal electrode arrays 14, 14′ are posterior of proximal ends of the distal bodies 17 of the medial temporal electrode arrays and the burr holes 20, 22. As such, as shown in FIG. 7C, the medial temporal electrode arrays 14, 14′ will no longer be substantially parallel with each other. Because of the different orientation between the two electrode arrays 14, 22, the angle φ between the medial temporal electrode array and the anterior polar electrode array may differ from the pattern of FIGS. 3A-3C. In the embodiment illustrated in FIGS. 10A-10C, the angle φ may be between about 70 degrees and about 120 degrees.

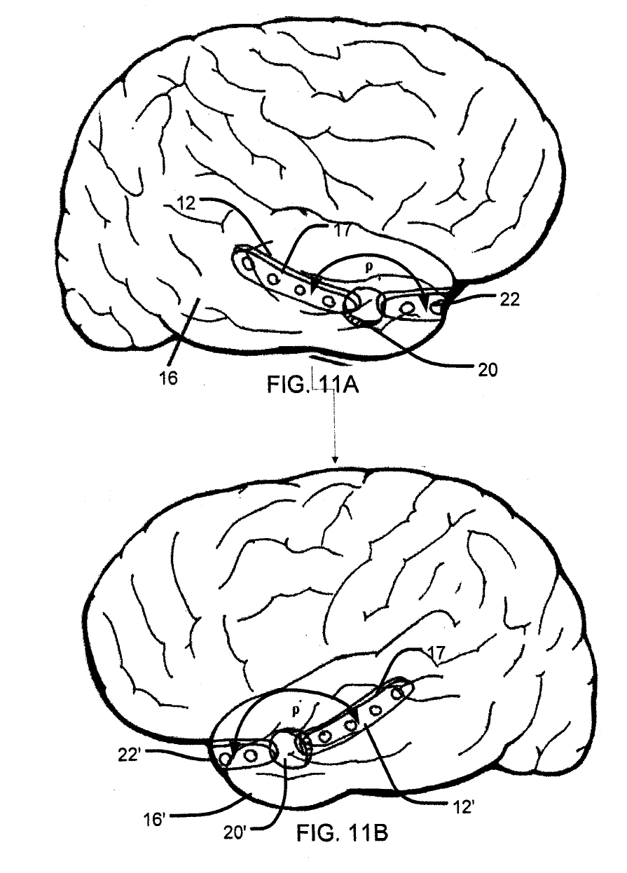

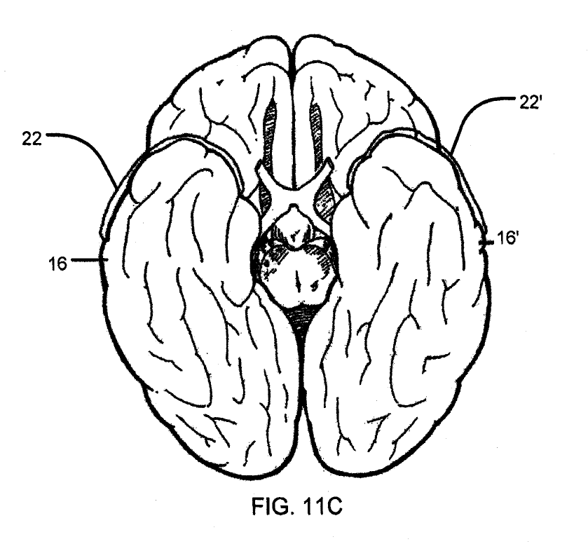

FIGS. 11A-11C illustrate lateral temporal electrode arrays 12, 12′ and anterior polar electrode arrays 22, 22′ that have been inserted into a patient with more anteriorly positioned burr holes 20, 20′. As such, the lateral temporal electrode arrays 12, 12′ do not extend as far posteriorly as in the embodiment of FIGS. 6A-6C, and the anterior polar electrode arrays 22, 22′ extend farther around the anterior polar portions of the temporal lobes.

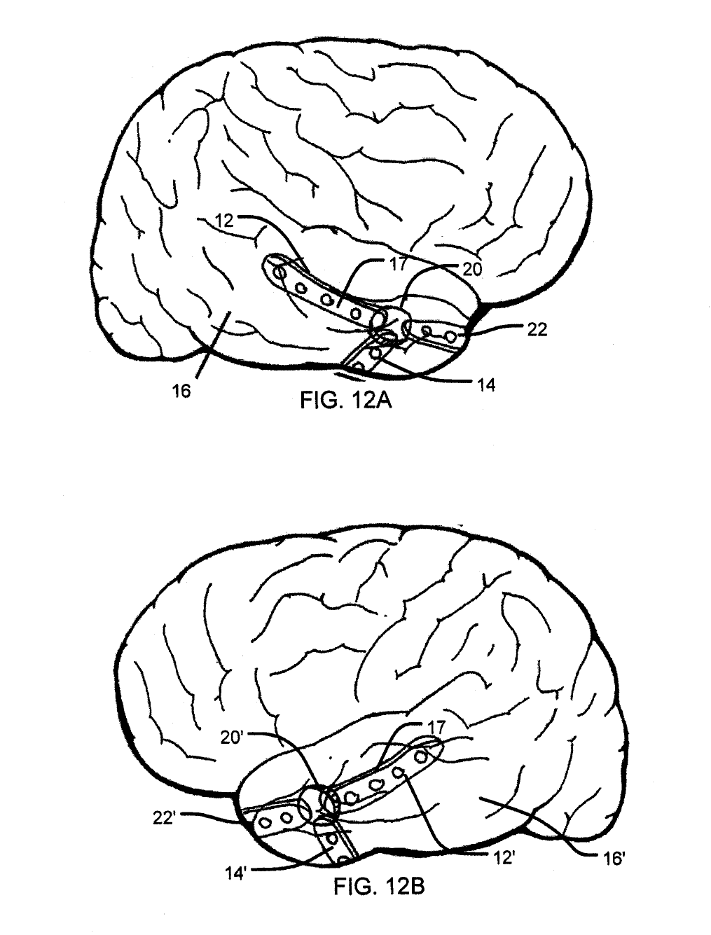

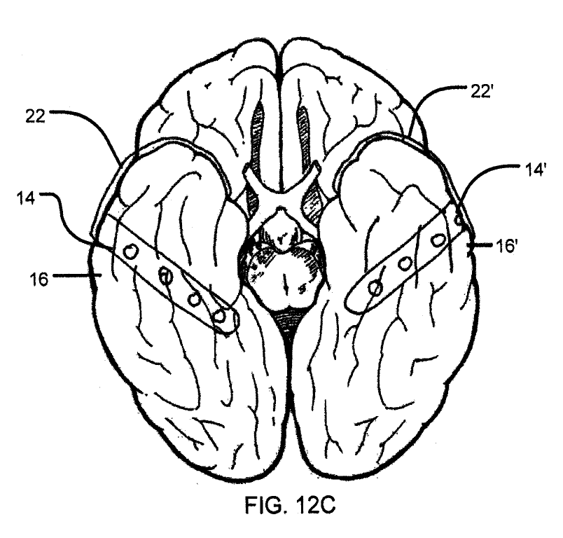

FIGS. 12A-12C illustrate another electrode array pattern, which includes lateral temporal strip electrode arrays 12, 12′, medial temporal strip electrode arrays 14, 14′ that extend posteriorly along the temporal lobes from more anterior burr holes 20, 20′ and anterior polar electrode arrays 22, 22′ that wrap around the anterior surfaces 24, 24′ of the temporal lobes 16, 16′. The relative orientations between each of the electrode arrays 12, 14, 22 will be similar to the embodiments described above in FIGS. 9A-11C.

While FIGS. 2A-12C illustrate separate array elements in the electrode array pattern, it should be appreciated that the electrode arrays themselves may be physically attached to each other. In one embodiment the four electrode arrays which are shown in FIGS. 2-4 are composed of two sets of two bifurcated distal bodies having a single leads and connectors.

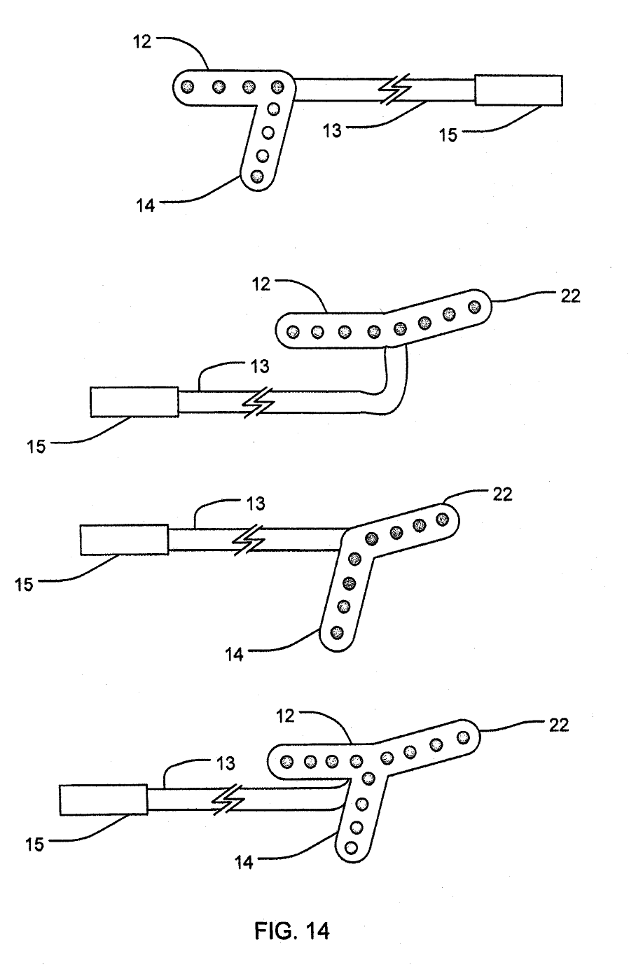

FIG. 13 illustrates two electrode arrays 210 having four electrodes 215 each being combined as a single bifurcated assembly 220 and connector 225. Alternatively, other embodiments may form a single electrode array formed as a bifurcated or trifurcated distal body. For example, FIG. 14 illustrates some examples of the integrally formed arrays that form the electrode array pattern. The single unit electrode array pattern has a number of advantages. First, the single unit would maintain the desired orientation between the legs (12, 14, 22) of the electrode array pattern and would be able to minimize movement of the legs, relative to each other. Second, by integrating the various electrode arrays, a single lead body 13 and connector assembly 15 could be used for the electrode array pattern, which reduces the likelihood of a lead failure and makes tunneling of the leads to the subclavicular pocket easier.

In addition to using the electrode contacts for sampling brain activity signals from the patient's temporal lobe, it may also be desirable to use one or more of the electrode contacts of the arrays to deliver electrical stimulation to the patient's temporal lobe. The electrical stimulation may be delivered through all of the electrode contacts or only selected electrode contacts. For example, it may be desirable to deliver electrical stimulation only to the electrode contacts that are adjacent the portion of the temporal lobe that is generating the abnormal brain activity. Thus, if different portions of the brain generate the abnormal activity over time, different electrode contact may be used to treat the patient. In other embodiments, the arrays may include a distal tip of a catheter (not shown) that may be used for delivering a drug to the patient's brain.

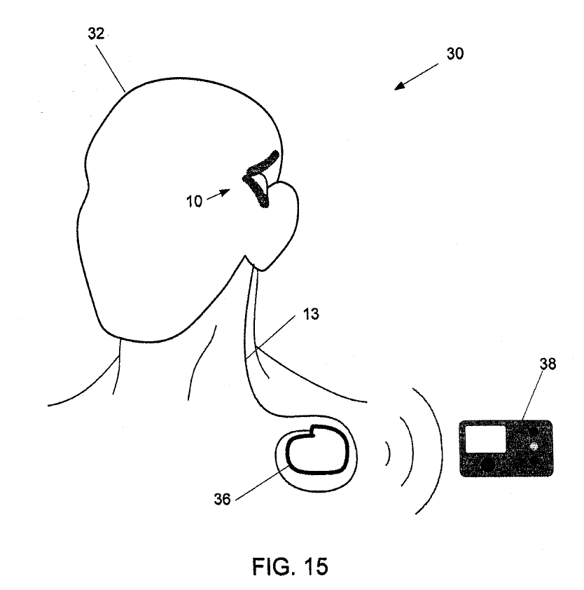

FIG. 15 illustrates an example of a system 30 for which the electrode array pattern 10 of the present invention may be used. The system 30 can be used to monitor a neurological condition of patient 32 for purposes of estimating a patient's propensity for transitioning into an ictal state. The system 30 of the illustrated embodiment provides for substantially continuous sampling and analysis of brain wave electrical signals.

The system 30 typically comprises one or more predetermined electrode array patterns 10, an implanted communication assembly (or communication unit 36) in communication with the one or more electrode arrays, and an external assembly 38 that is positioned outside the subject's body. The electrode array is shown electrically joined via lead body 13 to a communication unit 36, but could be in wireless communication with the communication unit or other external devices. The electrode array preferably functions to detect brain activity and are preferably implanted into the brain, subdurally, epidurally, partially or fully in the skull, or between the skull and one or more layers of the patient's scalp, but preferably do not include depth electrodes. The electrode array is preferably implanted through a single opening (not shown), such as a burr hole, created in the skull. The electrode array is preferably a predetermined dispersion pattern 10 (in some embodiments a predetermined dispersion pattern that includes multiple electrode arrays) which is preferably dependent upon a prior determination of a brain region in which an epileptic focus resides. The individual electrodes of the electrode array are preferably spaced between 10 mm and 20 mm from an adjacent electrode on the same array.

The implanted communication assembly or communication unit 36 is preferably configured to sample the brain activity signal with the electrode array. The external assembly 38 is preferably configured to receive a data signal from the implanted communication assembly and to indicate a subject's susceptibility to a seizure.

In one embodiment, the lead body 13 and the communication unit 36 will be implanted in the patient. For example, the communication unit 36 may be implanted in a sub-clavicular or abdominal cavity of the patient. In alternative embodiments, the lead body 13 and communication unit 36 may be implanted in other portions of the patient's body (e.g., in the head) or attached to the patient externally.

The communication unit 36 may be configured to facilitate the sampling of brain signals from the electrodes. Sampling of brain activity is typically carried out at a rate above about 200 Hz, and preferably between about 200 Hz and about 1000 Hz, and most preferably at or above about 400 Hz. The sampling rates could be higher or lower, depending on the specific features being monitored, the patient, and other factors. Each sample of the patient's brain activity is typically encoded using between about 8 bits per sample and about 32 bits per sample, and preferably about 16 bits per sample. In alternative embodiments, the communication unit 36 may be configured to measure the signals on a non-continuous basis. In such embodiments, signals may be measured periodically or aperiodically.

An external device 38 may be carried external to the body of the patient. The external device 38 can receive and store signals, including measured brain signals and possibly other physiological signals, from the communication unit 36. Communication between the external device 38 and the communication unit 36 (or the wireless electrodes) may be carried out through wireless communication, such as a radiofrequency link, infrared link, optical link, ultrasonic link, or other conventional or proprietary wireless link. The wireless communication link between the external device 38 and the communication unit 36 may provide a one-way or two-way communication link for transmitting data.

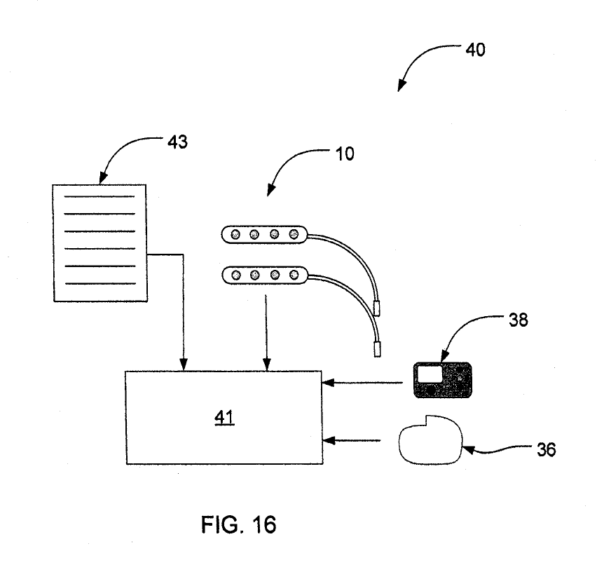

FIG. 16 illustrates an example of a packaged system or kit 40 that is embodied by the present invention. The packaged system 40 may include a package 41 that includes one or more compartments for receiving one or more electrode arrays 10 and instructions for use (IFU) 43 that describe any of the methods described herein. If desired, the kit 40 may also include the implantable communication unit 36 and/or the external unit 38. Such components may be in their own packaging (not shown) or in a compartment within the package 41.

A more detailed description of systems and algorithms that may use the electrode array are described in commonly owned U.S. Pat. Nos. 6,366,813; 6,819,956; 7,209,787; 7,242,984; 7,277,758; 7,231,254; 7,403,820; 7,324,851; 7,623,928; U.S. patent application Ser. Nos. 11/321,897, filed Dec. 28, 2005; 11/321,898, filed Dec. 28, 2005; 11/322,150, filed Dec. 28, 2005; 11/766,742, filed Jun. 21, 2007; 11/766,751, filed Jun. 21, 2007; 11/766,756, filed Jun. 21, 2007; 11/766,760, filed Jun. 21, 2007; 12/020,507, filed Jan. 25, 2008; 11/599,179, filed Nov. 14, 2006; 12/053,312, filed Mar. 21, 2008; 12/020,450, filed Jan. 25, 2008; 12/035,335, filed Feb. 21, 2008; and 12/180,996, filed Jul. 28, 2008, the complete disclosures of which are incorporated herein by reference.

The methods of the present invention are directed toward methods of implanting a plurality of electrodes to form a predetermined electrode array pattern on a surface of the patient's brain. In a preferred embodiment, the method is directed toward implanting an electrode array substantially over a seizure foci or seizure network to monitor brain signals that are predictive or indicative of a patient's susceptibility of a seizure (e.g., seizure prediction or seizure detection) and/or to deliver stimulation to the patient's brain. The electrode array may, however, be used for any other type of system or method that is used to monitor brain activity.

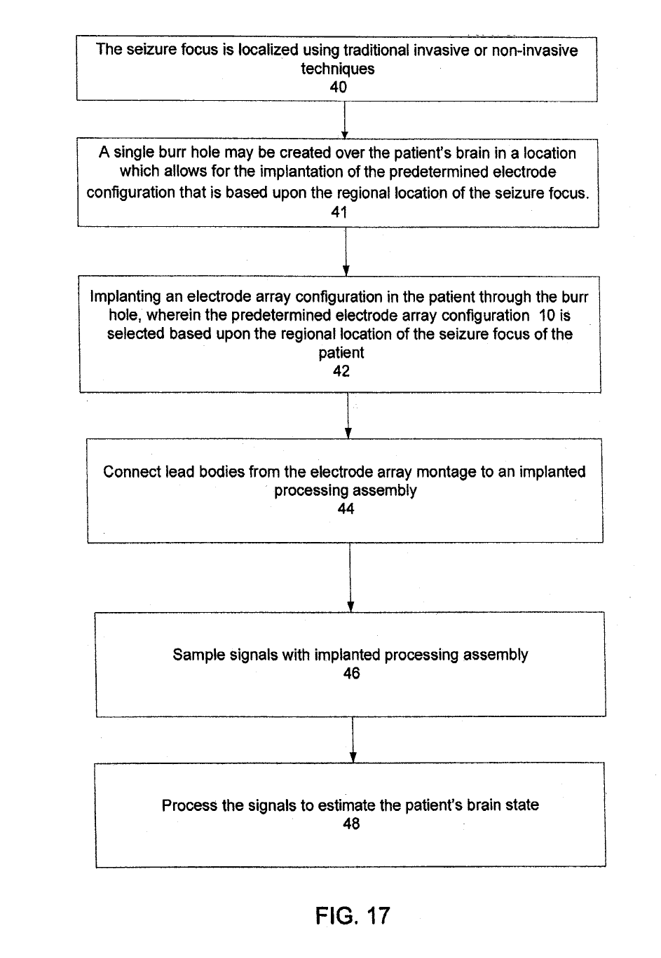

FIG. 17 illustrates one example of a method that is embodied by the present invention. At step 40, the lobe or lobes of the brain in which the patient's seizures are thought to originate are identified, for example, the seizure focus can be localized using traditional invasive or non-invasive techniques. Alternatively, identifying the lobe or lobes of the brain may not require localization of a seizure focus. At step 41 a single burr hole may be created over the patient's brain in a location which allows for the implantation of the predetermined electrode array pattern that is based upon the regional location of the seizure focus. If bilateral implantation is desired, a single burr hole may be created over each of the temporal lobes. Of course, for embodiments that are minimally invasive, the electrode array may be formed outside the skull and the burr hole (and step 40) is not needed.

At step 42, a predetermined electrode array pattern 10 is selected based upon the regional location of the seizure focus of the patient and implanted into the patient and preferably positioned on the surface of the dura mater, but may alternatively be positioned in any other suitable location of the brain, such as underneath the dura mater and on the cortical surface of the patient's brain. The electrode array may be selected such that at least one of the electrodes is in proximity to the seizure focus. In one embodiment, 32 or fewer electrodes are positioned in a dispersed pattern on the surface of the brain of the patient. The dispersed pattern is preferably dimensioned such that it cannot be circumscribed by a circle having a radius of 4 cm projected onto the brain surface. At step 44, the lead body may be threaded back through the burr hole and tunneled down to an implanted processing assembly—which may be in the cranium, a sub-clavicular pocket, or an abdominal pocket—and the connector assembly is connected to a corresponding connector assembly in the implanted processing assembly (i.e., a seizure advisory system).

At step 46, the implanted processing assembly is used to sample brain activity signals with the electrode array pattern. The implanted processing assembly may process the signal as described above. At step 48, the sampled signals may be processed to estimate the patient's brain state. The processing of the sampled signals may be performed in the implanted processing assembly, in an external device (see, e.g., FIG. 11) that is in wireless communication with the implanted processing assembly, or in a combination thereof. As described above, the brain state may be indicative of the patient's propensity and/or susceptibility for a seizure. Such methods are also applicable to estimating brain states of the other neurological or psychiatric disorders described above.

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. For example, while the above describes that each of the electrode arrays include a small number of macroelectrodes, it may be desirable to have some or all of the electrode contacts be microelectrodes so as to enable monitoring of one or more single neurons or small groups of neurons. Furthermore, while not shown in the figures, one or more of the contacts on the electrode array may be replaced with a catheter tip that may be used to deliver a local dosage of a pharmacological agent (e.g., antiepileptic drug) directly to the patient's brain. It is intended that the following claims define the scope of the invention and that methods and structures within the scope of these claims and their equivalents be covered thereby.

What is claimed is:

1. A method of monitoring brain activity signals in a patient, the method comprising:
identifying a lobe or lobes of the brain in which the patient's seizures originate;
selecting an electrode array from a plurality of predetermined dispersed electrode array patterns based on the identified lobe or lobes of the brain;
implanting the electrode array within the patient's cranium to place the electrodes in contact with the identified lobe or lobes of the brain; and
coupling electrodes to a seizure advisory system.

2. The method of claim 1 wherein identifying the lobe or lobes of the brain comprises identifying a lobe or lobes of the brain in which the patient's seizures originate without localization of a seizure focus.

3. The method of claim 1 wherein the electrode array comprises a furcated electrode substrate.

4. The method of claim 1 wherein the implanting step comprises implanting the electrode array to cover a portion of a cortical surface of the brain which cannot be circumscribed by a radius of 4 cm.

5. The method of claim 1 wherein the lobe or lobes of the brain comprise the frontal and temporal lobe, wherein implanting the electrode array comprises:
implanting a first electrode array parallel to an interhemispheric fissure in the patient;
implanting a second electrode array spanning between a precentral sulcus to an anterior edge of a temporal pole;
implanting a third electrode array spanning between the precentral sulcus to the temporal pole; and
implanting a fourth electrode array spanning between the precentral sulcus to a posterior portion of a Sylvian Fissure.

6. The method of claim 5 further comprising surgically exposing part of a skull and creating a single opening approximately 1 cm anterior to the precentral sulcus and 1 cm lateral to the interhemispheric fissure and implanting the electrode arrays through the opening.

7. The method of claim 5 wherein at least one electrode array comprises 4 separate electrodes which are linearly spaced 20 mm apart from adjacent electrodes of the same array.

8. The method of claim 1 where the lobe or lobes of the brain comprise the temporal lobe wherein implanting the plurality of electrodes comprises:
implanting a first electrode array spanning from a middle temporal gyrus in line with a spinal cord to a temporal pole;
implanting a second electrode array is inserted mesially from the middle temporal gyrus in line with the spinal cord and wrapping under the temporal lobe;
implanting a third electrode array spanning from the middle temporal gyrus in line with the spinal cord posteriorly along the middle temporal gyrus; and
implanting a fourth electrode array spanning from the middle temporal gyrus in line with the spinal into the temporal lobe both anteriorly and superiorly, passing approximately perpendicular to a Sylvian fissure.

9. The method of claim 8 further comprising surgically exposing part of the skull and creating a single opening over the middle temporal gyrus in line with the spinal cord and implanting the electrode arrays through the opening.

10. The method of claim 8 wherein the first, second and third electrode arrays each comprise 4 separate electrodes which are linearly spaced 10 mm apart from adjacent electrodes of the same array and the fourth electrode array comprises 4 separate electrodes which are linearly spaced 20 mm apart from adjacent electrodes of the same array.

11. The method of claim 1 where the lobe or lobes of the brain comprise the temporal and parietal lobes, wherein implanting the plurality of electrodes comprises;
implanting a first electrode array spanning between a precentral sulcus to an anterior edge of a temporal pole;
implanting a second electrode array spanning between the precentral sulcus the temporal pole;
implanting a third electrode array spanning between the precentral sulcus to a posterior portion of the Sylvian Fissure; and
implanting a fourth electrode array spanning from the precentral sulcus posteriorly, approximately parallel to an interhemispheric fissure, projecting towards an occipital lobe.

12. The method of claim 11 further comprising surgically exposing part of the skull and creating a single opening approximately 1 cm anterior to the precentral sulcus and 1 cm lateral to the interhemispheric fissure and implanting the electrode arrays through the opening.

13. The method of claim 11 wherein each electrode array comprises 4 separate electrodes which are linearly spaced 20 mm apart from adjacent electrodes of the same array.

14. An EEG electrode array comprising:
a substrate; and
a plurality of electrodes comprising no more than 32 electrodes disposed on the substrate;
the array being configured to be inserted through a single opening in a patient cranium and dispersed on a cortical surface of the brain radially away from the opening.

15. The electrode array of claim 14 wherein the electrodes are dispersed in a radial pattern larger than a circle having a radius of 4 cm.

16. The electrode array of claim 14 wherein the plurality of electrodes comprises 16 electrodes.

17. The electrode array of claim 14 further comprising two lead bodies, each lead body comprising:
a proximal end configured to couple to an implantable seizure advisory system; and
a bifurcated distal region having a first and second branch,
each branch having an electrode array comprising 4 electrodes.

18. The electrode array of claim 14 wherein the EEG electrode array is configured for implantation within a frontal and temporal lobe and comprises four electrode arrays, comprising:
a first electrode array adapted to be implanted parallel to an interhemispheric fissure in the patient;
a second electrode array adapted to span between a precentral sulcus to an anterior edge of a temporal pole;
a third electrode array adapted to span between the precentral sulcus to the temporal pole; and
a fourth electrode array adapted to span between the precentral sulcus to a posterior portion of a Sylvian Fissure.

19. The electrode array of claim 14 wherein the EEG electrodes are configured for implantation within a temporal lobe and comprises four electrode arrays, comprising:
a first electrode array adapted to span from a middle temporal gyrus in line with a spinal cord to a temporal pole;
a second electrode array adapted to be inserted mesially from the middle temporal gyrus in line with the spinal cord and wrapping under the temporal lobe;
a third electrode array adapted to span from the middle temporal gyrus in line with the spinal cord posteriorly along the middle temporal gyrus; and
a fourth electrode array adapted to span from the middle temporal gyrus in line with the spinal cord into the temporal lobe both anteriorly and superiorly, passing approximately perpendicular to a Sylvian fissure.

20. The electrode array of claim 14 wherein the EEG electrodes are configured for implantation within a temporal and parietal lobes comprising:
a first electrode array adapted to span between a precentral sulcus to an anterior edge of a temporal pole;
a second electrode array adapted to span between the precentral sulcus the temporal pole;
a third electrode array adapted to span between the precentral sulcus to a posterior portion of the Sylvian Fissure; and
a fourth electrode array adapted to span from the precentral sulcus posteriorly, approximately parallel to an interhemispheric fissure, projecting towards an occipital lobe.

21. A seizure advisory system comprising:
an electrode array having fewer than 32 electrodes in a predetermined dispersed pattern adapted to be implanted through a single opening in the skull of a patient and deployed in the predetermined dispersed radial pattern to detect a brain activity signal;
a communication assembly adapted to be implanted in the patient in communication with the electrode array, the communication assembly being configured to sample the brain activity signal with the electrode array;
an external assembly adapted to be positioned outside the patient's body and to receive a data signal from the communication assembly to indicate the patient's susceptibility to a seizure.

22. A seizure advisory system of claim 21 wherein the opening is a burr hole.

23. A seizure advisory system of claim 21 wherein the electrode array has no depth electrodes.

24. A seizure advisory system of claim 21 wherein spacing between adjacent electrodes within the same array is between 10 and 20 mm.

25. The seizure advisory system of claim 21 wherein the electrode array is configured for implantation within a frontal and temporal lobe of the patient's brain, wherein the electrode array comprises:
a first electrode array adapted to be implanted parallel to an interhemispheric fissure;
a second electrode array adapted to span between a precentral sulcus to an anterior edge of a temporal pole;
a third electrode array adapted to span between the precentral sulcus to the temporal pole; and
a fourth electrode array adapted to span between the precentral sulcus to a posterior portion of a Sylvian Fissure.

26. The seizure advisory system of claim 21 wherein the electrode array is configured for implantation within a temporal lobe of the patient's brain, wherein the electrode array comprises:
a first electrode array adapted to span from a middle temporal gyms in line with a spinal cord to a temporal pole;
a second electrode array adapted to be inserted mesially from the middle temporal gyrus in line with the spinal cord and to wrap under the temporal lobe;
a third electrode array adapted to span from the middle temporal gyrus in line with the spinal cord posteriorly along the middle temporal gyms; and
a fourth electrode array adapted to span from the middle temporal gyrus in line with the spinal into the temporal lobe both anteriorly and superiorly, passing approximately perpendicular to a Sylvian fissure.

27. The seizure advisory system of claim 21 wherein the EEG electrode array is configured for implantation within temporal and parietal lobes of the patient's brain, wherein the electrode array comprises:
a first electrode array adapted to span between a precentral sulcus to an anterior edge of a temporal pole;
a second electrode array adapted to span between the precentral sulcus and the temporal pole;
a third electrode array adapted to span between the precentral sulcus to a posterior portion of the Sylvian Fissure; and
a fourth electrode array adapted to span from the precentral sulcus posteriorly, approximately parallel to an interhemispheric fissure, to project towards an occipital lobe.

28. A method of positioning electrodes for monitoring brain activity signals in a patient, the method comprising:
identifying a lobe or lobes of the patient's brain in which the patient's seizures originate;
positioning 32 or fewer electrodes in a dispersed pattern on a surface of the brain such that the disperse pattern cannot be circumscribed by a circle having a radius of 4 cm projected onto the brain surface; and
coupling the plurality of electrode arrays to a seizure advisory system.

29. The method of claim 28 wherein identifying the lobe or lobes of the brain comprises identifying a lobe or lobes of the brain in which the patient's seizures originate without localization of a seizure focus.

30. The method of claim 28 wherein identifying the lobe or lobes of the brain comprises locating a seizure focus within the lobe or lobes of the brain, and positioning the plurality of electrodes comprises ensuring that at least one of the plurality of electrodes is in proximity to the seizure focus.

31. The method of claim 28, wherein a first electrode and a second electrode of the plurality of electrodes are spaced greater than 8 cm apart.

32. The method of claim 28 further comprising processing signals from at least one of the plurality of electrodes to estimate the patient's susceptibility for a seizure.

33. The method of claim 28 further comprising wirelessly transmitting a signal from the seizure advisory system to an external device.

34. The method of claim 28 wherein a portion of the seizure advisory system is implanted within the patient's body.

35. The method of claim 28 wherein positioning the plurality of electrodes comprises inserting all of the electrodes through a single opening in the skull.

36. The method of claim 35 wherein the opening is a burr hole.

37. The method of claim 28 wherein positioning the plurality of electrodes comprises positioning the plurality of electrodes on dura mater of the patient's brain.

38. The method of claim 28 wherein positioning the plurality of electrodes comprises positioning the plurality of electrodes underneath dura mater and on a cortical surface of the patient's brain.

39. The method of claim 28 wherein positioning the plurality of electrodes comprises positioning two or more substrates supporting the plurality of electrodes in a dispersed pattern on the surface of the brain.

40. The method of claim 39 wherein the substrates are coupled to a common lead body adapted to connect to the seizure advisory system.
